(19) United States
(12) Patent Application Publication
(10) Pub. No.: US 2010/0076019 A1
(43) Pub. Date: Mar. 25, 2010

(54) ALCOHOL FREE FORMULATION OF ARGATROBAN

(75) Inventor: Nageswara R. Palepu, South Hampton, PA (US)

Correspondence Address: LUCAS & MERCANTI, LLP, 475 PARK AVENUE SOUTH, 15TH FLOOR, NEW YORK, NY, 10016, US

(73) Assignee: EAGLE PHARMACEUTICALS, INC., Woodcliff Lake, NJ (US)

(21) Appl. No.: 12/442,975

(22) PCT Filed: Oct. 9, 2007
(86) PCT No.: PCT/US07/21533

§ 371 Date: Mar. 26, 2009

Related U.S. Application Data
(60) Provisional application No. 60/850,725, filed on Oct. 11, 2006.

(30) Foreign Application Priority Data
Sep. 26, 2007 (US) 11904067

(51) Int. Cl. A61K 31/4709; A61P 3/00

(52) U.S. Cl. 514/314

(57) ABSTRACT

An aqueous formulation of argatroban and of related compounds is disclosed along with a reconstitutable formulation, each of which is substantially, if not totally alcohol free. The formulations are also substantially free, if not totally free, of mono-, di-, and oligo-saccharides. An especially preferred embodiment is a ready-to-use 1 mg/ml injectable dosage form having argatroban, lactobionic acid, and methionine.

### CROSS-REFERENCE TO RELATED APPLICATIONS

This application is a continuation-in-part of a US Application by the same inventor under the same title, filed Sep. 26, 2007 and claims the benefit of priority of U.S. Provisional Application Ser. No. 60/850,725, filed Oct. 11, 2006 and U.S. Provisional Application Ser. No. 60/847,556, filed Sep. 27, 2006. This application is also a continuation-in-part of co-owned PCT/US07/20725, filed Sep. 26, 2007 having the same title and inventorship as the instant application and claiming priority from the same two provisional applications above and to co-owned US application filed concurrently herewith having the same inventorship and title as the present application. Each of these applications as well as each patent and patent application mentioned in the rest of this specification is incorporated herein by reference in their entireties.

### STATEMENT REGARDING FEDERALLY SPONSORED RESEARCH OR DEVELOPMENT

Not Applicable

### FIELD OF THE INVENTION

The present invention relates to argatroban and related compounds and to the solubilization thereof to yield injectable and other aqueous solutions of desired concentration in aqueous media without the need for alcohols or other solvents and/or without the use of saccharides.

### BACKGROUND OF THE INVENTION

Argininesulfonamides are known to have anti-thrombotic activities (see e.g., Japanese Patent No. 1382377). However, it is very difficult to obtain a solution containing any of the argininesulfonamides at high concentrations due to their general poor solubility in water. Therefore these compounds are generally not suitable for use in injection formulations containing them at high concentrations. U.S. Pat. No. 5,214,052 attempts to solve this problem by dissolving these compounds in a dissolution media containing water, ethanol, and a saccharide (inclusive of monosaccharides, disaccharides, oligosaccharides and their reduced sugar alcohol counterparts). Argatroban, currently marketed by Encysive in the US is sold as a 2.5 ml vial of 100 mg/ml argatroban concentrate having 750 mg D-sorbitol, and 1000 mg dehydrated alcohol per ml, which concentrate is subsequently diluted to 1 mg/ml argatroban for actual use. While that formulation allows for advantages in packaging and dissolution to final concentration, it suffers from the drawback of having ethanol present in a not insignificant amount, especially when the patient in question is of smaller body weight. Current administration rates include 6 ml/hr (of the 1 mg/ml diluted solution) for a 50 kg patient to 17 ml/hr (of the 1 mg/ml diluted solution) for a 140 kg patient each for the duration of the procedure for which argatroban administration is desired. Thus, each vial supplied provides 250 ml of administrable diluted solution, resulting in substantial waste of material in all but the most prolonged procedures (250 ml being sufficient for over 40 hours for a 50 kg patient and over 14 hours for a 140 kg patient).

### OBJECT OF THE INVENTION

An object of the invention is to provide a method for improving the solubility of argininesulfonamides in a completely aqueous system, in particular avoiding the use of organic solvents such as monoalcohols of 1-4 carbon atoms, especially ethanol, and still obtain solutions of sufficient concentration for use in parenteral administration.

A further object of the invention is to provide an argininesulfonamide formulation that is substantially free of saccharides, inclusive of mono-saccharides, di-saccharides, oligosaccharides, and their corresponding sugar alcohols.

Another object of the invention is to provide a dosage form of argininesulfonamide which is not as concentrated so that further dilution for use does not result in substantial waste of material in most typical administration settings.

A further object of the invention is to provide a dosage form of argininesulfonamide not requiring an extensively large dilution, yet be concentrated sufficiently to be convenient for preparing for use and less subject to dissolution errors than with current marketed argatroban.

Still a further object of the invention is to provide 1 mg/ml ready to administer solutions of argininesulfonamide in 5 ml to 500 ml vials and 25 ml to 500 ml infusion bags.

Yet another object of the invention is to provide an argininesulfonamide ready-to-administer formulation having a storage stability of at least about 18 months.

Still another object of the invention is to provide an argininesulfonamide ready-to-administer formulation having a substantial stability with respect to pH in a terminal sterilization operation.

An even further object of the invention is to provide an argininesulfonamide ready-to-administer formulation having a substantial stability with respect to degradation product in a terminal sterilization operation.

An even further object of the invention is to provide an argininesulfonamide ready-to-administer formulation having a substantial stability with respect to degradation product in the presence of an antioxidizing agent such as methionine in an aseptic operation or in a terminal sterilization operation.

Still another object of the invention is the use of lactobionic acid as a solubilizer and/or stabilizer to enhance the aqueous solubility as well as stability of argininesulfonamide.

Yet another object of the invention is to provide an argatroban ready to administer aqueous solution for injection having carbonate and/or bicarbonate ion present.

Still another object of the invention is to provide an argininesulfonamide aqueous formulation that can be incorporated into non-injectable dosage forms inclusive of ointments, creams, suppositories, liquid fill tablets, liquid fill capsules, and transdermal devices, among others.

Still another object of the invention is the use of lactobionic acid as a solubilizer and/or stabilizer to enhance the aqueous solubility as well as stability of argininesulfonamide.

Yet further objects of the invention will be apparent to those of ordinary skill in the art.

### SUMMARY OF THE INVENTION

The invention provides a method for dissolving argininesulfonamides comprising dissolving an argininesulfonamide (most preferably argatroban) in an aqueous, preferably, buffered system substantially free of lower alcohols and in the substantial absence of saccharides (mono-, di-, and oligo-saccharides and their corresponding sugar alcohols). Further, the invention provides pharmaceutical compositions containing the argininesulfonamides.

### DETAILED DESCRIPTION OF THE INVENTION

In certain embodiments, the invention provides a method for dissolving an argininesulfonamide comprising dissolving an N²-arylsulfonyl-L-arginine having general formula (I) or a pharmaceutically acceptable slat thereof

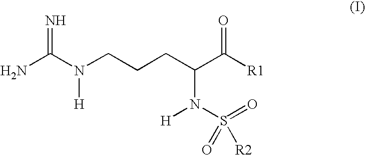

wherein R¹ represents an unsubstituted or substituted 2-carboxypiperidino group (where there may be up to 5 substituents independently selected from alkyl, carboxy, an amidated carboxy (the amidated carboxy nitrogen being further unsubstituted or having one or two alkyl substituents that may be joined so as to form a 5, 6, or 7 membered ring with the amidated carboxy nitrogen), an esterified carboxy, or a pharmaceutically acceptable salt of the carboxy group), preferably a 4-alkyl-carboxypiperidino group, more preferably a (2R, 4R)-4-alkyl-2-carboxypiperidino group. The alkyl group herein is a lower alkyl having 1 to 5 carbon atoms such as, without limitation, methyl, ethyl, propyl, isopropyl and butyl. Preferably, R¹ represents a (2R, 4R)-4-methyl-2-carboxypiperidino group.

R² represents a phenyl group or a condensed polycyclic compound residue. The condensed polycyclic compound residue defined herein includes a benzene ring that binds to the sulfur atom of the sulfonyl group and is condensed with one or more other rings, which other rings may be carbocyclic or heterocyclic and which further has 3 to 14 carbon atoms as the ring-constituent atoms (exclusive of the ring atoms in the benzene ring that is attached to the sulfonyl sulfur atom). The benzene ring included in the condensed polycyclic compound residue binds to the sulfur atom of the sulfonyl group in the general formula (I) at any position of the benzene ring, provided that the position on the benzene ring binding to the sulfur atom is not particularly limited. The heteroatom or heteroatoms constituting the heterocyclic ring may be oxygen, nitrogen or sulfur atoms. Other than the benzene ring directly bound to the sulfonyl sulfur in general formula (I) above, the other rings may be aromatic, or partially saturated, and in the case of a tricyclic group, the third ring not bound to the benzene ring may further be fully saturated.

The heteroring nitrogens may be further unsubstituted or further substituted with an alkyl, and the heteroring sulfur atoms may be unoxidized, mono-oxidized, or di-oxidized (i.e., —S—, —S(O)—, or —SO₂—). Preferably, the condensed polycyclic compound residue is a dicyclic compound residue including benzene ring condensed with one other ring, preferably one five- or six-membered ring which may be heterocyclic or a tricyclic compound residue inclusive of the benzene ring condensed with two other rings, preferably two rings of five or six-members each which may be heterocyclic. Examples of such condensed polycyclic compound residues include anthryl, phenanthryl, benzofuranyl, dibenzothienyl, phenoxthinyl, quinolyl, carbazolyl, acridinyl, phenazinyl, phenothiazinyl, phenoxazinyl, benzimidazolyl, fluorenyl, 2,3-dihydrobenzofuranyl, thioxathenyl, naphthyl, tetrahydronaphthyl, isoquinolyl, tetrahydroquinolyl and tetrahydroisoquinolyl.

If desired, R² can be substituted with one or more substituents selected from lower alkyl groups, lower alkoxy groups and lower alkyl-substituted amino groups. The lower alkyl group is alkyl group having 1 to 5 carbon atoms, such as, without limitation, methyl, ethyl, propyl, isopropyl, butyl, isobutyl and tert-butyl. The lower alkoxy group is alkoxy group having 1 to 5 carbon atoms, such as, without limitation, methoxy, ethoxy, propoxy, isopropoxy and butoxy. The lower alkyl-substituted amino group may be unsubstituted or further mono or di-substituted, each of such substituents being selected from lower alkyl having 1 to 5 carbon atoms, such as, without limitation, methyl, ethyl, propyl, isopropyl, butyl, isobutyl and tert-butyl.

Preferably, R² represents a 3-methyl-1,2,3,4-tetrahydro-8-quinolyl group.

Furthermore, unless specifically limited in the text or the context requires otherwise, any compound within the scope of formula I above that has one or more chiral centers is deemed to include the individual optical isomers as well as mixtures of those isomers, and any compound within the scope of formula I that is indicated as having chiral centers, whether or not referred to as an optical isomer, includes each of the individual optical isomers as well as mixtures thereof in any proportions, and any compound within the scope of formula I that is identified as an optical isomer includes reference to the other optical isomers and mixtures thereof in various proportions.

Examples of argininesulfonamides suitable for use in the invention include the following compounds and the pharmaceutically acceptable salts of each:
(2R, 4R)-1-[N²-(3-isopropoxybenzenesulfonyl)-L-arginyl]-4-methyl-2-piperidinecarboxylic acid;
(2R, 4R)-1-[N²-(3,5-dimethyl-4-propoxybenzenesulfonyl)-L-arginyl]-4-methyl-2-piperidinec arboxylic acid;
(2R, 4R)-1-[N²-(5,6,7,8-tetrahydro-2-naphthalenesulfon)-yl)-L-arginyl]-4-methyl-2-piperi dinecarboxylic acid;
(2R, 4R)-1-[N²-(5-dimethylamino-1-naphthalenesulfonyl)-L-arginyl]-4-methyl-2-piperidinec arboxylic acid;
(2R, 4R)-1-[N²-(3-methyl-1,2,3,4-tetrahydro-8-quinoline-sulfonyl)-L-arginyl]-4-methyl-2-piperidinecarboxylic acid;
(2R, 4R)-1-[N²-(2-dibenzothiophenesulfonyl)-L-arginyl]-4-methyl-2-piperidinecarboxylic acid;
(2R, 4R)-1-[N²-(2,4-dimethoxy-3-butoxybenzenesulfonyl)-L-arginyl]-4-methyl-2-piperidinec arboxylic acid;
(2R, 4R)-1-[N²-(3,5-dimethyl-4-propoxybenzenesulfonyl)-L-arginyl]-4-methyl-2-piperdinecarboxylic acid;
(2R, 4R)-1-[N²-(3-ethyl-1,2,3,4-tetrahydro-8-quinoline-sulfonyl)-L-arginyl]-4-methyl-2-piperidinecarboxylic acid;
(2R, 4R)-1-[N²-(2-carbazolesulfonyl)-L-arginyl]-4-methyl-2-piperidinecarboxylic acid;
(2R, 4R)-1-[N²-(2-fluorenesulfonyl)-L-arginyl]-4-methyl2-piperidinecarboxylic acid;
(2R, 4R)-1-[N²-(2-phenoxthinesulfonyl)-L-arginyl]-4-methyl-2-piperidinecarboxylic acid;
(2R, 4R)-1-[N²-(2-anthracenesulfonyl)-L-arginyl]-4-methyl-2-piperidinecarboxylic acid; and
(2R, 4R)-1-[N²-(7-methyl-2-naphthalenesulfonyl)-L-arginyl]-4-methyl-2-piperidinecarboxylic acid;

as well as their 4-ethyl analogues, their 4-propyl analogues, their 4-butyl analogues and their 4-pentyl analogues; as well as the pharmaceutically acceptable salts thereof, whether acid addition salts (e.g., hydrochloride salts of the basic nitrogens in the compounds) or basic salts (e.g., amine salts of the sulfonamide group and/or the carboxy group). Most preferably, the argininesulfonamide used in the present invention is argatroban or a pharmaceutically acceptable salt thereof.

The invention can use the salts of argininesulfonamides having the general formula (I). The salts may be acid addition salts (there being a sulfonamide group and in many of the compounds an additional carboxy group present) prepared by reacting the argininesulfonamide of general formula (I) with any pharmaceutically acceptable inorganic or organic acid such as, without limitation, hydrochloric acid, hydrobromic acid, hydroiodic acid, sulfuric acid, nitric acid, phosphoric acid, acetic acid, citric acid, maleic acid, succinic acid, lactic acid, tartaric acid, gluconic acid, glucuronic acid, ethers of glucuronic acid or gluconic acid (such as lactobionic acid), benzoic acid, methanesulfonic acid, ethanesulfonic acid, benzenesulfonic acid and p-toluenesulfonic acid. Further, the salts may be inorganic or organic salts prepared by reacting the argininesulfonamide of general formula (I) with any pharmaceutically acceptable organic or inorganic bases such as sodium hydroxide, potassium hydroxide, ammonium hydroxide, triethylamine, procaine, dibenzylamine, N,N′-dibenzylethylenediamine and N-ethylpiperidine.

In one method for dissolving an argininesulfonamide according to the invention, the argininesulfonamide and/or its salt is dissolved in an amino acid aqueous solution. The amino acid used in the invention is preferably selected from arginine, glycine, methionine, or other amino acids with at least one basic group pKa >9.0 or mixtures thereof. The amino acid can be used as the acid or a salt thereof or mixtures thereof. While either D- or L- or D,L-amino acids can be used, L-amino acids are generally preferred in this first embodiment. In another embodiment, discussed below, D,L-amino acids are generally preferred. The pH of the drug and amino acid solution is adjusted to about 8.0 to about 10.0 with one or more pharmaceutically acceptable carboxylic acids such as, without limitation, acetic acid or any other carboxylic acid or dicarboxylic acid or hydroxy carboxylic acid; and may be adjusted with either the acid itself, or a salt thereof, or mixtures thereof if an appropriate pH can be reached using such salt or mixture of salt and acid. Preferred amino acids are arginine, glycine, and methionine. In this first embodiment arginine and glycine are more highly preferred, while in a second embodiment, discussed more specifically below, methionine is a more preferred amino acid. The amino acid, when having buffering capacity in the desired region, can be self-buffering so that a separate buffer is not required; in this case pH adjustment can be accomplished with inorganic acids and bases (e.g., HCl or NaOH, among others). In addition to the monocarboxylic amino acids such as arginine, glycine, and methionine, among others, the amino acid may be a dicarboxylic amino acid selected from formulas II and III

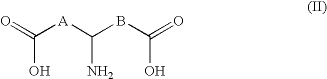

where A and B are each independently selected from (a) a branched or unbranched alkyl of 0-6 carbons, provided that the sum of the carbon atoms in A and B do not exceed 6; (b) a branched or unbranched heteroalkyl having up to 2 heteroatoms selected from N and O, provided that the sum of the carbon and heteroatoms in A and B do not exceed 6; (c) 5-6 membered cycloalkyl, 5-6 membered heteroalkyl having up to 2 heteroatoms selected form N, O, and S; (d) 6 membered aryl or heteroaryl having up to 2 hetero atoms selected from N, O, and S; preferably dicarboxylic amino acids of formula II are those where A and B are both limited to alkyls of 0-6 carbons, more preferably those where the amino group is in an alpha or beta position to at least one of the carboxylic groups, and even more preferably where the two carboxylic acid groups are separated by at least 2 carbon atoms.

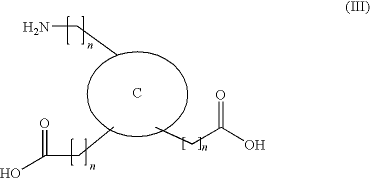

where ring C is a six-membered aromatic or heteroaromatic ring or partially or fully saturated analog thereof, having up to 2 heteroatoms selected from N, O, and S and on which the amino and the carboxylic acid groups can be present at any available position of such ring C, and each n is independently an integer of 0-3, preferably each n is zero. Exemplary dicarboxylic amino acid of formula II are, without limitation, glutamic acid and aspartic acid. An exemplary amino acid with formula III is amino phthalic acid. In all of the amino acids for the present invention, the pKa of the basic function (or of at least one basic function if more than one is present) should be greater than about 9.

In the present invention, reference to a given weight of a compound that can exist as a salt or in free form is with reference to the free form of the compound. Thus, for a compound such as argatroban having a molecular weight of 526 Dalton and monosodium argatroban having a molecular weight of 548 Dalton, and argatroban monohydrochloride having a molecular weight of 561 Dalton, an indication of “52.6 mg of argatroban” will mean 52.6 mg of argatroban non-salt form, or if the monosodium salt is being discussed, 52.6 mg of argatroban will mean 54.8 mg of monosodium argatroban having 52.6 mg of the argatroban moiety present, or if the argatroban monohydrochloride is being discussed, it will mean 56.1 mg of argatroban monohydrochloride having 52.6 mg o the argatroban non-salt moiety present. Corresponding calculations to find the exact weight of the salt form under discussion for other salts are known to those of ordinary skill in the art. Weights of amino acids will also be referenced to the non-salt forms thereof with appropriate calculations to find the precise weight of a particular salt being known to those of ordinary skill in the art.

The amino acid is generally present in amounts that are about 1.5 to about 2.5, preferably about 2 times the amount of the compound of formula (I) present (based on the non-salt form of the compound of formula (I)). When present, the carboxylic acid (other than carbonic acid salt), is generally present in amounts that are about 1.5 to about 2.5, preferably about 2 times the amount of the compound of formula (I) present (based on the non-salt form of the compound of formula (I)), while (when present) the carbonic acid salt is generally present in an amount of about 3.0 to about 9.8 (based on CO₃) times the amount of the compound of formula (I) present (based on the non-salt form of the compound of formula (I)), preferably about 1.4 to about 5.2 times, more preferably about 1.5 to about 2.5 times, more preferably about 1.9 to about 2.0 times when the amino acid is absent and preferably about 3.0 to about 5.2 times, more preferably about 4.1 to about 4.2 times when the amino acid is present. Those of ordinary skill in the art will be able to adjust these amounts for the situation where both a carboxylic acid (other than carbonic acid salt) is present in combination with a carbonic acid salt.

Water as used in the present invention (unless indicated otherwise or the context requires otherwise) includes aqueous injectable fluids including, but not limited to distilled water, purified water, water for injection, a physiological saline, Ringer's Solution, Lactated Ringer's Solution and 5% dextrose solution, and any of these may be used for diluting concentrates. Preferably in preparing the ready-to-administer formulations, distilled water, purified water or water for injection is used with a post formulation sterilization procedure, or preferably water for injection is used if post-formulation sterilization is not being used.

The manner of how to dissolve the argininesulfonamide having the general formula (I) in water and optionally in an amino acid aqueous solution is not particularly limited. Generally, the amino acid (or its salt or a mixture of the amino acid and its salt), when present, is dissolved in water and then the pH is adjusted upward, if need be, to about pH 8.7 to about 10 with the addition of inorganic or organic base (or salts of carboxylic acid or mixtures of its salts and their conjugate acids or conjugate bases (inclusive of alkali metal salt(s) and ammonium salts of carbonic acid)) thereto followed by mixing. These two steps can be reversed if desired. Next, the argininesulfonamide is slowly added while stirring until complete dissolution. If desired, but not required, the pH can then be adjusted downward. Where concentrates are to be made for subsequent dilution, higher pH can be tolerated for the dissolution and storage phases as the subsequent dilution will bring the pH closer to physiologic pH before injection. Concentrates having the amino acid in the range of 50 mg/ml (especially when using arginine as the amino acid) will have a pH as high as about 11 to about 11.5 before addition of the argininesulfonamide. In these concentrates, the argininesulfonamide is dissolved and the pH is adjusted downward into the range of about 8.7 to 9.5 as discussed above using an appropriate acid or buffer. Where a ready-to-administer injection formulation is desired, the ready-to-administer product pH should generally not be greater than about pH about 9.5, preferably not greater than about 9.2, and more preferably is usually about pH 8.7, about 8.8, about 8.9, about 9.0, about 9.1, or about 9.2. Certain advantageous ready to use formulations of the present invention have a pH of about 8.7, while certain other advantageous ready-to-use formulations of the present invention have a pH of about 9.2.

The temperature on dissolution is not particularly limited. When the argininesulfonamide is dissolved in water, however, it is preferable to warm the water to about 40° C. to about 80° C. for accelerating the dissolution rate, about 70° C. being advantageous in a number of situations.

The concentration of argininesulfonamide in the solution can be selected within a wide range depending on the intended uses. According to the invention, the solution in which the argininesulfonamide is dissolved may result in concentrations of the argininesulfonamide that are several folds higher than the concentrations of the argininesulfonamide typically obtained with the solubility of the argininesulfonamide in water alone. Most advantageously for one embodiment of the concentrates of the present invention, when argatroban or a pharmaceutically acceptable salt thereof is the active agent, the argatroban can be dissolved up to (based on an equivalent of non-salt form argatroban) about 7.5 mg/ml, preferably about 6 mg/ml, most preferably about 5 mg/ml. An additional embodiment within the invention is a ready-to-administer solution of about 0.8 to about 1.25 mg/ml, preferably about 0.9 to about 1.1 mg/ml, more preferably about 1 mg/ml compound of formula (I). All amounts presented are amounts of compound of formula (I) free compound, that is the non-salt. Corresponding amounts of various salts will be readily known to those of ordinary skill in the art by routine calculation.

In a second embodiment of the invention, the argininesulfonamide compound of formula (I), preferably argatroban, or a pharmaceutically acceptable salt thereof is dissolved in an aqueous solution of a gluconic or glucuronic acid (or a sugar ether of either, where the sugar position 1 is etherified with one of the hydroxyl groups of the gluconic or glucuronic acid, preferably the ether is lactobionic acid or a salt thereof) and/or with an alkali metal (e.g., sodium or potassium) or ammonium salt, preferably a sodium salt, of carbonic acid (e.g., sodium carbonate, sodium bicarbonate, and mixtures thereof) and optionally an amino acid or salt thereof. In each case, the salts, if present are pharmaceutically acceptable salts, and in the case of use of the solution as an injectable, the salt is compatible with its use in an injectable formulation. Preferably, the formulation comprises a compound of formula (I) or a pharmaceutically acceptable salt thereof; a gluconic acid or glucuronic acid, or an ether of either or a pharmaceutically acceptable salt thereof (preferably lactobionic acid or a pharmaceutically acceptable salt thereof) and/or an alkali metal or ammonium salt of carbonic acid, preferably a sodium salt of carbonic acid or mixture of sodium salts of carbonic acid; optionally an amino acid, preferably an anti-oxidant amino acid (more preferably methionine), arginine, or glycine, either in the D-, L-, or D,L-form, preferably as the D,L-form, the amino acid optionally in the form of a pharmaceutically acceptable salt thereof; and water (which water may further contain an optional inert osmolarity adjuster (other than a saccharide) so as to bring the solution to an appropriate osmolarity if desired); and wherein the formulation is substantially free of ethanol, preferably being substantially free of at least one of if not both of (a) monohydric alcohols having 1 to 4 carbons; and (b) mono-, di-, and oligosaccharides and their corresponding sugar alcohols. For the present invention, “substantially free” when referring to a lower alcohol means less than about 5% v/v, preferably less than about 2.5%, more preferably less than about 1%, more preferably less than about 0.5% v/v; while when referring to “saccharide” means less than about 10% w/v, preferably less than about 7.5%, more preferably less than about 5%, still more preferably less than about 2.5%, even more preferably less than about 1%, yet more preferably less than about 0.05% w/v. Formulations having the required components and either or both lower alcohols and saccharides (inclusive of mono-, di- and oligo-saccharides and their corresponding sugar alcohols) are within the present invention, should one so desire, but the emphasis of the invention is primarily on the avoidance of the lower alcohol or saccharide and particularly the avoidance of both. Inclusion of methionine or another anti-oxidant amino acid improves the product stability especially with respect to terminal sterilization, and is therefore one particularly preferred embodiment.

In this embodiment, it is preferable to first heat the water, preferably to boiling, then allow the water to cool to a temperature of about 30-50° C., preferably about 35° C. The carboxylic acid (preferably lactobionic acid or pharmaceutically acceptable salt thereof and/or alkali metal salt or ammonium salt of carbonic acid) is added and dissolved. Then, any optional amino acid is added and dissolved. Then the compound of formula (I) (or a pharmaceutically acceptable salt thereof) is added and dissolved. In this procedure, the amino acid and carboxylic acid addition steps can be reversed, if desired or the amino acid can be added after the compound of formula (I). The pH is adjusted as convenient at any point prior to the addition of the compound of formula (I) or pharmaceutically acceptable salt thereof to a pH in excess of about 8.5, preferably in excess of about 8.6, more preferably to about pH 8.7 to about 9.5, more particularly about pH 8.7 to about 9.2, still more preferably about 8.7, about 8.8, about 8.9, about 9.0, about 9.1 or about 9.2, so as to aid in the dissolution of the compound of formula (I).

Higher pH's are acceptable for the dissolution phase for concentrate formulations that will be further diluted before actual injection, provided the dilution brings down the pH to a range such that upon dilution to the final use concentration the pH is physiologically acceptable for injection purposes, typically less than about 9.5, generally less than about 9.2, preferably less than about 9.0, more preferably less than about 8.8, still more preferably about 8.7; diluted pH of about 8.6, about 8.7, about 8.8, about 8.9, about 9.0, about 9.1, and about 9.2 are all acceptable if so desired. If need be, final adjustment of pH can be made with an acid or base or buffer as appropriate such as among others hydrochloric acid, sodium hydroxide, or a buffer solution of either or both the carboxylic acid/carboxylic acid salt (inclusive of blends of alkali metal or ammonium salts of carbonic acid) and/or the amino acid/amino acid salt. Thus, a concentrate formulation may be prepared within the instant invention which has a substantially high pH, while the ready-to-use formulations will have a generally weakly alkaline pH, generally greater than about 8.6 and generally less than about 9.2.

The solution thus obtained containing any of the argininesulfonamides having the general formula (I), the amino acid, water and carboxylic acid constitutes a first embodiment of the pharmaceutical composition of the invention, while the solution containing (a) the compound of formula (I); (b) water; (c) (1) gluconic acid, glucuronic acid, and/or ether thereof, and/or (c)(2) an alkali metal or ammonium salt or mixtures of alkali metal or ammonium salts of carbonic acid; and (d) optional (preferably anti-oxidant) amino acid constitutes a second embodiment, and the solution containing the argatroban, amino acid, and water with or without the carboxylic acids constitutes a third embodiment of the invention. As will be readily recognized, embodiments one and two overlap when the carboxylic acid in the first embodiment is selected from gluconic acid, glucuronic acid, the ether of either (especially lactobionic acid) and alkali metal or ammonium salt or mixture of alkali metal or ammonium salts of carbonic acid; and the amino acid in the first embodiment is an anti-oxidant amino acid.

The pharmaceutical compositions of the invention are useful for treating thrombosis and for treating and/or prophylaxis of any other condition for which the active agents are already known to be useful. Accordingly, the pharmaceutical compositions can be used as anti-thrombotic agents.

The pharmaceutical composition of the invention may contain antiseptic, anti-oxidant, soothing agents and the like. And, if necessary any pharmaceutical ingredient(s) other than the argininesulfonamides may be added to form a combined preparation, provided such other ingredient is not unacceptable for the indication and route of administration; however, the invention compositions and processes are generally substantially free of, if not totally free of (1) ethyl alcohol, more preferably any lower alcohol of 1-4 carbon atoms or (2) a saccharide, preferably a monosaccharide or disaccharide or oligo-saccharide, more preferably any saccharide (wherein saccharide herein optionally includes the reduced sugar alcohol counterparts thereto), or (3) both (1) and (2). While formulations within the invention may contain either or both of the alcohols or saccharides (in more than insignificant amounts, they are preferably substantially free, more preferably totally free of one or both of these materials as stated earlier.

The primary composition of the invention in the first embodiment is a pharmaceutical injectable and is administered as an injection. This injectable composition may further contain stabilizer, buffer, preservative and the like, which are acceptable for injection. If desired, the injectable composition according to the invention is prepared to contain an argininesulfonamide at a high concentration, which is used by diluting with water, electrolyte (e.g., normal saline, among others), carbohydrate solution (e.g., 5% Dextrose), Ringer's solution or the like at or close to the time of administration (such as by infusion and/or dialysis). The concentrated formulation may contain amounts of up to about 7.5 mg (based on free argininesulfonamide (non-salt form), preferably up to about 5 mg per ml. In the case of argininesulfonamide, this is generally diluted for administration to about 1 mg (based on argininesulfonamide non-salt form) per ml. Dilution of the concentrate to other concentrations for use as an injection will be within the ordinary skill in the art. The formulation, as detailed further below, can also be prepared as a lyophilizate or as a sterile dry fill product that can be reconstituted with appropriate diluent diluent to either the concentrate concentrations of this embodiment or to the ready-to-use concentrations in the second embodiment.

The primary composition of the invention second embodiment is also as a pharmaceutically acceptable injection formulation, primarily as a ready-to-administer composition. In this embodiment, the argininesulfonamide (or salt thereof based on free argininesulfonamide), preferably argatroban, is present in a concentration of no more than about 1.25 mg/ml, preferably about 1.1 mg/ml, more preferably about 1 mg/ml, in a pH of about 8.5 to about 9.2, preferably about 8.6 to about 8.9, more preferably about pH 8.7 to about 8.9 in a water solution containing (a) as a carboxylic acid, at least one member selected from (1) gluconic acid, glucuronic acid, and sugar ethers thereof, especially lactobionic acid and pharmaceutically acceptable salts thereof and/or (2) alkali metal salt or ammonium salt or mixture of alkali metal salts and/or ammonium salts of carbonic acid, and optionally (b) at least one (preferably anti-oxidant) amino acid or pharmaceutically acceptable salt thereof. If the free argatroban concentration is greater than about 1.0, the concentrate can be diluted with sufficient water of a suitable pH, with or without the amino acid or the carboxylic acid. In embodiments in which an osmotic adjuster material is utilized (other than as part of dilution at the point of administration), the osmotic adjuster is any injectably suitable osmotic adjuster but preferably is not a saccharide or sugar alcohol, especially when an alcohol of 1-4 carbon atoms is present in the formulation.

Alternatively, in other embodiments, the pharmaceutical composition of the invention is a solution for topical application, an ointment or a cream (in which the solution is taken up by an appropriate topical ointment or cream base), or a suppository (in which the solution is taken up by an appropriate suppository base). When the pharmaceutical composition is used as the solution for topical application, the solution prepared above can be used as it is, or upon dilution (the concentration for use is not limited to 1.0 mg/ml). The solution (typically concentrated) can also be used in the preparation of a transdermal product for local or systemic administration of argatroban. When used for transdermal administration, the transdermal dosage form is one generally capable of containing a solution such as (without limitation) a reservoir type transdermal having (without limitation) an active agent reservoir defined by a space between an impermeable backing, and a permeable skin contacting layer which itself is covered by a removable impermeable layer (prior to use). Alternatively, the transdermal dosage form may contain the dissolved argatroban in a monolithic layer which may or may not further contain adhesive in such monolithic layer and which monolithic layer is covered by a removable impermeable layer (prior to use). At the time of administration of the transdermals, the impermeable removable layer is removed and the remainder of the device is applied to the patient's skin allowing drug to flow into the patient's skin.

### EXAMPLES

The invention will now be further described by the following, non-limiting examples.

### Example 1

(2R, 4R)-1-[N²-(3-methyl-1,2,3,4-tetrahydro-8-quinolinesulfonyl)-L-arginyl]-4-methyl-2-piperidinecarboxylic acid (argatroban) was dissolved (to a concentration of 5 mg/ml) in an aqueous system containing 50 mg/ml arginine with pH of the final solution adjusted to 9.2 with acetic acid. The dissolution of argatroban was carried out at 25° C.

### Example 2

Argatroban is dissolved (to a concentration of 5 mg/ml) in an aqueous system containing 50 mg/ml arginine with pH adjusted to 9.0 with acetic acid. The dissolution of argatroban is carried out at 50° C.

### Example 3

Argatroban is dissolved (to a concentration of 5 mg/ml) in an aqueous system containing 50 mg/ml arginine with pH adjusted to 9.0 with tartaric acid. The dissolution of argatroban is carried out at 25° C.

### Example 4

Argatroban is dissolved (to a concentration of 5 mg/ml) in an aqueous system containing 50 mg/ml arginine with pH adjusted to 9.0 with citric acid. The dissolution of argatroban is carried out at 25° C.

### Example 5

Argatroban is dissolved (to a concentration of 5 mg/ml) in an aqueous system containing 50 mg/ml arginine with pH adjusted to 9.0 with adipic acid. The dissolution of argatroban is carried out at 25° C.

### Example 6

Argatroban is dissolved (to a concentration of 5 mg/ml) in an aqueous system containing 50 mg/ml lysine with pH adjusted to 9.0 with acetic acid. The dissolution of argatroban is carried out at 25° C.

### Example 7

Argatroban is dissolved (to a concentration of 5 mg/ml) in an aqueous system containing 50 mg/ml arginine with pH adjusted to 10.0 with acetic acid. The dissolution of argatroban is carried out at 25° C.

### Example 8

Argatroban is dissolved (to a concentration of 5 mg/ml) in an aqueous system containing 50 mg/ml arginine with pH adjusted to 8.0 with acetic acid. The dissolution of argatroban is carried out at 25° C.

### Example 9

Examples 1-8 are repeated except that the dissolution is carried out at the following concentrations (based on argatroban non-salt form) using the temperatures and form of argatroban as indicated:

| | Concentration of | |
|---|---|---|
| Form | argatroban moiety | Temperature |
| Non-salt | 5 mg/ml | 15° C. |
| Non-salt | 5 mg/ml | 20° C. |
| Non-salt | 5 mg/ml | 30° C. |
| Non-salt | 5 mg/ml | 35° C. |
| Non-salt | 7.5 mg/ml | 15° C. |
| Non-salt | 7.5 mg/ml | 20° C. |
| Non-salt | 7.5 mg/ml | 30° C. |
| Non-salt | 7.5 mg/ml | 35° C. |
| Sodium Salt | 5 mg/ml | 15° C. |
| Sodium Salt | 5 mg/ml | 20° C. |
| Sodium Salt | 5 mg/ml | 25° C. |
| Sodium Salt | 5 mg/ml | 30° C. |
| Sodium Salt | 5 mg/ml | 35° C. |
| Sodium Salt | 7.5 mg/ml | 15° C. |
| Sodium Salt | 7.5 mg/ml | 20° C. |
| Sodium Salt | 7.5 mg/ml | 25° C. |
| Sodium Salt | 7.5 mg/ml | 30° C. |
| Sodium Salt | 7.5 mg/ml | 35° C. |
| Hydrochloride salt | 5 mg/ml | 15° C. |
| Hydrochloride salt | 5 mg/ml | 20° C. |
| Hydrochloride salt | 5 mg/ml | 25° C. |
| Hydrochloride salt | 5 mg/ml | 30° C. |
| Hydrochloride salt | 5 mg/ml | 35° C. |
| Hydrochloride salt | 7.5 mg/ml | 15° C. |
| Hydrochloride salt | 7.5 mg/ml | 20° C. |
| Hydrochloride salt | 7.5 mg/ml | 25° C. |
| Hydrochloride salt | 7.5 mg/ml | 30° C. |
| Hydrochloride salt | 7.5 mg/ml | 35° C. |

### Example 10

Examples 1-9 are repeated except that the argatroban analog having the formula

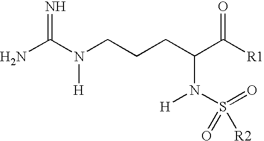

where R1 and R2 are selected as set forth in the table below is used and the mg amounts indicated in each prior Example is of the free compounds of this example rather than of argatroban:

| R1 | R2 |
|---|---|
| 2-carboxy-4-methyl-piperidin-1yl | 1,2,3,4-tetrahydro-8-quinolinyl |
| 2-carboxy-4-methyl-piperidin-1yl | 3-methyl-1,2,3,4-tetrahydro-7- |
| | quinolinyl |
| 2-carboxy-4-methyl-piperidin-1yl | 1,2,3,4-tetrahydro-7- |
| | quinolinyl |
| 2-carboxy-4-methyl-piperidin-1yl | 3-methyl-1,2,3,4-tetrahydro-6- |
| | quinolinyl |
| 2-carboxy-4-methyl-piperidin-1yl | 1,2,3,4-tetrahydro-6-quinolinyl |
| 2-carboxy-4-methyl-piperidin-1yl | 3-methyl-1,2,3,4-tetrahydro-5- |
| | quinolinyl |
| 2-carboxy-4-methyl-piperidin-1yl | 1,2,3,4-tetrahydro-5-quinolinyl |
| 2-carboxy-4-methyl-piperidin-1yl | 3-methyl-1,2,3,4-tetrahydro-8- |
| | quinolinyl |
| 2-carboxy-4-methyl-piperidin-1yl | 1,2-dihydro-8-quinolinyl |
| 2-carboxy-4-methyl-piperidin-1yl | 3-methyl-1,2-dihydro-7-quinolinyl |
| 2-carboxy-4-methyl-piperidin-1yl | 1,2-dihydro-7-quinolinyl |
| 2-carboxy-4-methyl-piperidin-1yl | 3-methyl-1,2-dihydro-6-quinolinyl |
| 2-carboxy-4-methyl-piperidin-1yl | 1,2-dihydro-6-quinolinyl |
| 2-carboxy-4-methyl-piperidin-1yl | 3-methyl-1,2-dihydro-5-quinolinyl |
| 2-carboxy-4-methyl-piperidin-1yl | 1,2-dihydro-5-quinolinyl |
| 2-carboxy-4-methyl-piperidin-1yl | 3-methyl-8-quinolinyl |
| 2-carboxy-4-methyl-piperidin-1yl | 8-quinolinyl |
| 2-carboxy-4-methyl-piperidin-1yl | 3-methyl-7-quinolinyl |
| 2-carboxy-4-methyl-piperidin-1yl | 7-quinolinyl |
| 2-carboxy-4-methyl-piperidin-1yl | 3-methyl-6-quinolinyl |
| 2-carboxy-4-methyl-piperidin-1yl | 6-quinolinyl |
| 2-carboxy-4-methyl-piperidin-1yl | 3-methyl-5-quinolinyl |
| 2-carboxy-4-methyl-piperidin-1yl | 5-quinolinyl |
| 2-carboxy-3-methyl-piperidin-1-yl | 3-methyl-1,2,3,4-tetrahydro-8- |
| | quinolyl |
| 2-carboxy-piperidin1-yl | 3-methyl-1,2,3,4-tetrahydro-8- |
| | quinolyl |
| 3-carboxy-piperiny-1-yl | 3-methyl-1,2,3,4-tetrahydro-8- |
| | quinolyl |
| 2-carboxy-5-methyl-piperidin-1-yl | 3-methyl-1,2,3,4-tetrahydro-8- |
| | quinolyl |
| 2-carboxy-4-methyl-piperidin-3-yl | 3-methyl-1,2,3,4-tetrahydro-8- |
| | quinolyl |
| 2-carboxy-3-methyl-piperidin-4-yl | 3-methyl-1,2,3,4-tetrahydro-8- |
| | quinolyl |
| 2-carboxy-piperidin-3-yl | 3-methyl-1,2,3,4-tetrahydro-8- |
| | quinolyl |
| 3-carboxy-piperiny-4-yl | 3-methyl-1,2,3,4-tetrahydro-8- |
| | quinolyl |
| 2-carboxy-5-methyl-piperidin-3-yl | 3-methyl-1,2,3,4-tetrahydro-8- |
| | quinolyl |
| 2-carboxy-5-methyl-pyridin-3-yl | 3-methyl-1,2,3,4-tetrahydro-8- |
| | quinolyl |

### Example 11

6 vials of 5 mg/ml argatroban solution of Example 1 are diluted to 1 mg/ml for a total of 30 ml of 1 mg/ml solution and used to administer the same to a 50 kg patient at the rate of 6 mg/hour for a procedure expected to be 4.5 hours. Upon completion of the procedure, there is a minimal amount of unused drug (less than 5 mg) as compared with over 200 mg that would result from the currently marketed argatroban 100 mg/ml 2.5 ml vials.

Attempts to use partial vials of the currently marketed 100 mg/ml 25 ml vial to dilute only 30 mg (0.3 ml) yield variations in actual amounts withdrawn for subsequent dilution and are thus not as reliable as using complete 5 mg/ml vials of the present invention.

### Example 12

The solution of Example 1 is taken up into a pharmaceutically acceptable cream or ointment base (such as Aquaphor) and used as a topical application form.

### Example 13

The solution of Example 1 is applied onto an impermeable backing layer and covered with a laminate made of a permeable membrane and a distally located impermeable release liner and the assemble is fused together to create a transdermally administrable dosage form of the solution of Example 1.

The solution of Example 1 is taken up into a polyethylene glycol 8000 base to prepare a suppository formulation of argatroban.

### Example 14

Water is heated to boiling and allowed to cool to about 35° C. Lactobionic acid is added thereto in an amount to achieve a concentration of about 2 mg/ml. Argatroban is added thereto in an amount sufficient to achieve a concentration of about 1 mg/ml. The solution is packaged in appropriate containers as a ready-to use injectable and the completed packages are terminally heat sterilized.

### Example 15

Example 14 is repeated except that the solution pH is adjusted to about 8.7 after addition of the lactobionic acid, but before the addition of the argatroban.

### Example 16

Example 14 is repeated except that the amounts of lactobionic acid and argatroban are increased by an additional 10% and after the argatroban has been dissolved, optionally additional water at pH about 8.7 is added to bring the final concentration of argatroban to about 1 mg/ml and the final concentration of lactobionic acid to about 2 mg/ml.

### Examples 17

Water was heated to boiling and allowed to cool to about 35° C. D,L-methionine was added to arrive at a methionine moiety concentration of about 2 mg/ml. Lactobionic acid was then added in an amount to achieve a concentration of about 2 mg/ml. Argatroban was then added thereto in an amount sufficient to achieve a concentration of about 1 mg/ml. The solution was packaged in appropriate containers as a ready-to use injectable and the completed packages were terminally heat sterilized.

### Examples 18-19

Examples 15-16 are repeated except that in Examples 18-19, DL-methionine is added before the lactobionic acid.

### Examples 20-25

Examples 16-16 are repeated except that in Examples 20-22, DL-methionine is added after the lactobionic acid, but before the argatroban; and in Examples 23-25, D,L-methionine is added after the argatroban, in each of Examples 23-25 in an amount to result in a concentration of about 2 mg/ml D,L-methionine, and in Examples 20-22 in an amount of about 10% greater than in Examples 17-19.

### Example 26

11 mg of argatroban was dissolved in 2.6 ml of 0.025 M sodium carbonate and the pH was adjusted with the addition of 1.5 ml of 0.025N HCl. Sufficient water for injection is added to bring the final solution volume to 10 ml, which has a pH of about 9.12.

### Examples 27-28

11 mg of argatroban is dissolved in 5.5 ml of 0.025N sodium carbonate. In Example 27, 20 mg of D,L-methionine is added and then the pH is adjusted downward with 1.5 ml of 0.025N HCl. In Example 28, these two steps are reversed. Water for injection is then added in a sufficient quantity to bring the final volume to 10 ml, which final solution has a pH of about 8.85.

### Example 29

Glycine was dissolved in water in an amount sufficient to achieve a glycine moiety final concentration of 2 mg/ml. The pH was then adjusted with 1N sodium hydroxide to about 9.2. Sufficient argatroban was added thereto to result in a final argatroban moiety concentration of about 1 mg/ml. The final pH was adjusted, if needed, to about 9.2 with 1N sodium hydroxide.

### Examples 30-34

Example 29 is repeated except that lactobionic acid is added in an amount to result in a final lactobionic acid concentration of about 2 mg/ml after the glycine is dissolved but before the pH is adjusted, and the pH is adjusted to 8.5 (Example 30), 8.7 (Example 31), 9.0 (Example 32), 9.5 (Example 33), or 10.0 (Example 34).

### Examples 35A-35E

Ready-to-use formulations of argatroban for injection having the following formulations are prepared having the following components:

| Formulation | Formul- | Formul- | Formul- | Formulation | Formulation |
|---|---|---|---|---|---|
| Component | ation A | ation B | ation C | D | E |
| Argatroban | 1 mg/ml | 1 mg/ml | 1 mg/ml | 1 mg/ml | 1 mg/ml |
| Lactobionic | 2 mg/ml | 2 mg/ml | 2 mg/ml | 2 mg/ml | — |
| Acid | | | | | |
| D,L- | — | 2 mg/ml | — | 2 mg/ml | — |
| methionine | | | | | |
| Glycine | 2 mg/ml | — | 2 mg/ml | 2 mg/ml | 2 mg/ml |
| pH | 9.2 | 8.7 | 8.7 | 8.7 | 9.5 |

### Examples 37-39

Example 35 A, C, D, and E is repeated is repeated except that the glycine is replaced by aspartic acid 2 mg/ml in Example 37, by glutamic acid in Example 38, and by aminophthalic acid in Example 39.

1. A pharmaceutically acceptable formulation of the compound of formula I or a pharmaceutically acceptable salt thereof,
which is solubilized in an aqueous solution at a concentration greater than that of the compound of formula I in water alone, with at least one of
(a) an optional amino acid;
(b) an optional a carboxylic acid selected from the group consisting of gluconic acid, glucuronic acid, gluconic acid ethers, glucuronic acid ethers, carbonic acid alkali metal salts, carbonic acid ammonium salts and mixtures thereof;
(c) an optional buffer
alkaline pH conditions 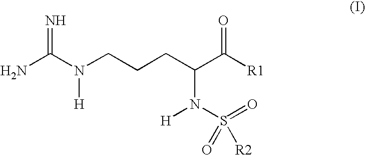
wherein
R¹ represents an unsubstituted or substituted 2-carboxypiperidino group (where there may be up to 5 substituents independently selected from alkyl, carboxy, an amidated carboxy (the amidated carboxy nitrogen being further unsubstituted or having one or two alkyl substituents which may me joined so as to form a 5, 6, or 7 membered ring with the amidated carboxy nitrogen), an esterified carboxy, or a pharmaceutically acceptable salt of the carboxy group,
R² represents a phenyl group or a condensed polycyclic compound residue, which residue includes a benzene ring which binds to the sulfur atom of the sulfonyl group and is condensed with one or more other rings which may be heterocyclic and which further has 3 to 14 carbon atoms as the ring-constituent atoms exclusive of those contained in the benzene ring attached to the sulfonyl sulfur atom, the heteroatoms being selected from nitrogen, oxygen, and sulfur, and in which said nitrogen atoms may be unsubstituted or substituted with lower alkyl and said sulfur atoms my be unoxidized, mono-oxidized, or deoxidized, said heterorings having from 1 to 4 heteroatoms, which formulation is substantially free of ethanol and substantially free of a mono-, di-, or oligo-saccharide and substantially free of a sugar alcohol.

2. The formulation of claim 1 which is (a) substantially free of ethanol and/or (b) substantially free of a mono-, di-, or oligo-saccharide and substantially free of a sugar alcohol.

3. The formulation of claim 1 in which said compound of formula I is present in a concentration equivalent to an amount (based on a non-salt form of said compound) selected from the group consisting of: about 1 mg/ml, about 1.25 mg/ml, about 2 mg/ml, about 2.5 mg/ml and about 5 mg/ml.

4. The formulation of claim 1 wherein said amino acid is selected from the group consisting of methionine, glycine, arginine, lysine, or any other amino acid where at least one of its basic group pKas is above 8.5, or mixtures thereof or a salt thereof, or a mixture said amino acid and said salt.

5. The formulation of claim 1 wherein said amino acid is arginine, glycine, or methionine.

6. The formulation of claim 1 wherein said buffer is at least one member selected from the group consisting of at least one of (1) a carboxylic acid, a hydroxy carboxylic acid, a dicarboxylic acid, with at least of its acid group pKa(s) greater than 3.0, a salt thereof, or a mixture of said carboxylic acid and said salt thereof and (2) an alkali metal or ammonium carbonate, alkali metal or ammonium bicarbonate, or mixtures thereof.

7. The formulation of claim 1 wherein said buffer is an acetate buffer, an amino acid buffer, or a lactobionic acid buffer, or a carbonate buffer.

8. The formulation of claim 1 wherein said amino acid is present in an amount of about 1 mg/ml to about 50 mg/ml.

9. The formulation of claim 1 wherein (1) the compound of formula (I) is argatroban or a pharmaceutically acceptable salt thereof or mixture thereof, (2) said amino acid is arginine, glycine, or methionine or a pharmaceutically acceptable salt thereof or mixture thereof, and (3) said buffer is an acetate buffer, lactobionic acid buffer, or alkali metal or ammonium carbonate/bicarbonate buffer.

10. The formulation of claim 1 packaged in a vial selected from 5 mg/vial to 500 mg/vial or in an IV infusion bag of a size selected from 25 ml/bag to about 500 ml/bag.

11. The argininesulfonamide formulation of claim 1 as a ready-to-administer aqueous solution wherein said compound of formula I is argatroban or a pharmaceutically acceptable salt thereof comprising
(a) argatroban or a pharmaceutically acceptable salt thereof in an amount of at least about 0.75 mg/ml (based on the argatroban moiety);
(b)(1) lactobionic acid or a pharmaceutically acceptable salt thereof in an amount (based on the non-salt form thereof) or a mixture of said lactobionic acid and lactobionic acid salt of at least about 1.5 times the weight of the argatroban (based on the argatroban moiety) and/or
(b)(2) an alkali metal or ammonium salt or mixture of alkali metal or ammonium salts of carbonic acid or mixture of lactobionic acid salts in an amount based on CO₃ of at least about 1.4 times the weight of the argatroban (based on the argatroban moiety); and
(c) optionally methionine or a pharmaceutically acceptable salt thereof in an amount (based on the non-salt form of methionine) of at least about 1.5 times the weight of the argatroban (based on the argatroban moiety).

12. The formulation of claim 11 wherein said argatroban or pharmaceutically acceptable salt thereof is present in an amount of about 0.75 mg/ml to about 1.25 mg/ml based on the non-salt form thereof.

13. The formulation of claim 11 wherein said lactobionic acid or pharmaceutically acceptable salt thereof is present (based on the non-salt form thereof) in an amount of not more than about 2.5 times the weight of the argatroban (based on the non-salt form thereof) and said alkali metal salt or mixture of alkali metal salts of carbonic acid in an amount based on CO₃ of not more than about 5.2 times the weight of the argatroban (based on the non-salt form of argatroban).

14. The formulation of claim 11 wherein said methionine or pharmaceutically acceptable salt thereof is present (based on the non-salt form thereof) in an amount of not more than about 2.5 times than weight of the argatroban (based on the non-salt form thereof).

15. The formulation of claim 11 having a pH in excess of 8.5.

16. The formulation of claim 15 having a pH in excess of about 8.6.

17. The formulation of claim 15 having a pH of about 8.7, about 8.8, about 8.9, about 9.0, about 9.1, or about 9.2.

18. The formulation of claim 11 having (1) a weight ratio of argatroban or pharmaceutically acceptable salt thereof: lactobionic acid or pharmaceutically acceptable salt thereof: methionine or pharmaceutically acceptable salt thereof (each based on the respective non-salt forms) of about 0.75 to about 1.25:about 1.50 to about 2.50:about 1.50 to about 2.50 or (2) a weight ratio of argatroban or pharmaceutically acceptable salt thereof: alkali metal or ammonium salt or mixture of alkali metal or ammonium salts of carbonic acid (based on CO₃): methionine or pharmaceutically acceptable salt thereof (each of the argatroban salt and amino acid salt based on the respective non-salt forms) of about 0.75 to about 1.25: about 1.4 to about 5.2: about 1.50 to about 2.50.

19. The formulation of claim 18, wherein said ratio is (a) about 1: about 2:about 2 when lactobionic acid is present and carbonic acid salts are absent, (b) about 1:about 1.9 to about 2.0:about 2 when carbonic acid salt is present and amino acids are absent, or (c) about 1: about 4.1 to about 4.2: about 2 when carbonic acid salt is present and amino acid is absent.

20. A pharmaceutical formulation of claim 1 further comprising a carrier selected from a cream base, an ointment base, a suppository base, liquid fill tablet components, liquid fill capsule components, or transdermal device components.

21. A reconstitutable formulation of a compound of formula (I) 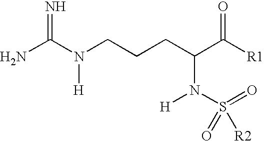
wherein
R¹ represents an unsubstituted or substituted 2-carboxypiperidino group (where there may be up to 5 substitutents independently selected from selected from alkyl, carboxy, an amidated carboxy (the amidated carboxy nitrogen being further unsubstituted or having one or two alkyl substituents which may me joined so as to form a 5, 6, or 7 membered ring with the amidated carboxy nitrogen), an esterified carboxy, or a pharmaceutically acceptable salt of the carboxy group),
R2 represents a phenyl group or a condensed polycyclic compound residue, which residue includes a benzene ring which binds to the sulfur atom of the sulfonyl group and is condensed with one or more other rings which may be heterocyclic and which further has 3 to 14 carbon atoms as the ring-constituent atoms exclusive of those contained in the benzene ring attached to the sulfonyl sulfur atom, the heteroatoms being selected from nitrogen, oxygen, and sulfur, and in which said nitrogen atoms may be unsubstituted or substituted with lower alkyl and said sulfur atoms my be unoxidized, mono-oxidized, or deoxidized, said heterorings having from 1 to 4 heteroatoms
comprising
(a) said compound of formula (I) or a salt thereof or mixtures thereof,
(b) optionally an amino acid or a salt thereof or mixtures thereof,
(c) at least one of (1) a non-amino acid carboxylic acid or salt thereof or mixtures thereof and (2) an alkali metal salt or ammonium salt of carbonic acid or mixtures thereof.

22. A method of reducing dosage administration errors in administering the compounds of formula (I) comprising providing a pharmaceutically acceptable concentrate formulation or a ready-to-use formulation of the compound of formula (I) as defined by the formulation of claim 1.

23. A method of reducing pharmaceutical active substance wastage in formulation of the compounds of formula (I) while simultaneously avoiding dosing errors introduced by use of partial vial usage, which method comprises providing the compound of formula (I) in a pharmaceutically acceptable dosage form according to claim 1.

24. A method of treating an argatroban treatable condition comprising administering to a patient having an argatroban treatable condition the composition of claim 1.

25. The method of claim 24 where said composition is in a ready-to-administer form.

26. The method of claim 24 where said composition is in the form of a concentrate and diluting said concentrate with an injectably suitable aqueous diluent to a suitable concentration for injection.
